(19) United States
(12) Patent Application Publication
(10) Pub. No.: US 2004/0242927 A1
(43) Pub. Date: Dec. 2, 2004

(54) Method for making adipic acid crystals and resulting crystals

(76) Inventors: Thierry Gisbert, Miribel (FR); Jean-Claude Masteal, Villeurbanne (FR)

Correspondence Address: Jean Louis Seugnet, Intellectual Property Department, Rhodia Inc CN 7500, 259 Prospect Plains Road, Cranbury, NJ, 08512-7500, US

(21) Appl. No.: 10/489,882

(22) PCT Filed: Sep. 17, 2002
(86) PCT No.: PCT/FR02/03167

(30) Foreign Application Priority Data
Sep. 18, 2001 (FR) 01/12039

(51) Int. Cl. C07C 55/02

(52) U.S. Cl. 562/590

(57) ABSTRACT

The present invention relates to a process for the manufacture of adipic acid crystals and to the crystals obtained. It relates more specifically to a process for the treatment of the adipic acid crystals obtained at the end of crystallization in order to obtain crystals which can be stored with minimum possibility of caking. This process consists of a maturing stage in order to partially remove the water present in the said crystals.

The present invention relates to a process for the manufacture of adipic acid crystals and to the crystals obtained.

It relates more specifically to a process for the treatment of the adipic acid crystals obtained at the end of crystallization in order to obtain crystals which can be stored with minimum possibility of caking.

Adipic acid is a major intermediate, in particular in the field of polymers and more particularly of polyamide and in the synthesis of polyurethanes. It is also used in numerous other applications, for example as adjuvants in detergent compositions.

Adipic acid is generally synthesized by oxidation by nitric acid of a cyclohexanone/cyclohexanol mixture in the presence of oxidation catalysts, such as vanadium and copper.

The adipic acid is recovered and purified by successive crystallization operations. This crystallization is generally carried out in an aqueous medium.

During these operations, the adipic acid is separated in particular from the other dicarboxylic acids formed, such as glutaric acid or succinic acid.

The adipic acid crystals produced at the end of crystallization are generally oblong crystals which can exhibit a highly irregular surface.

After washing and drying, these crystals are generally stored in kegs, bags or large size containers and are possibly transported to the site of their use, for example plants for the manufacture of polyamide or of hexamethylenediamine adipate salt.

During this storage and possibly transportation, caking, that is to say adhesion of several crystals to one another, often takes place. This phenomenon is highly damaging as it greatly reduces the flowability of the adipic acid during the feeding of the crystals in plants, for example for the manufacture of polyamides.

U.S. Pat. No. 5,471,001 provides a specific process for the crystallization of adipic acid with the use of ultrasound. The crystals obtained exhibit a better flowability and a reduced possibility of “caking” during storage and transportation.

Provision has also been made, in French Patent Application 2 795 721, for a process for the treatment of the crystals in order to modify the surface of the latter in order to reduce the ability to cake.

These various processes require the use of treatment stages which impose significant capital costs and which can lead to a loss in adipic acid yield.

One of the aims of the present invention is to provide a process which makes possible the manufacture of adipic acid crystals exhibiting a minimum possibility of caking when they are stored, in order to obtain good flowability during the charging or discharging of the containers or receptacles in which they are present.

To this end, a first subject-matter of the invention is adipic acid crystals, obtained by crystallization or treatment in an aqueous medium, which can be stored in containers or receptacles which are impermeable to external moisture for variable periods of time, without risk of caking, agglomeration or adhesion of the crystals. With the adipic acid crystals of the invention, it is easy to empty the storage or transportation containers. The use of the adipic acid is, for this reason, greatly facilitated.

According to the invention, the adipic acid crystals are characterized in that the content of exchangeable water in the crystals is less than or equal to 100 ppm, preferably less than 50 ppm.

The term “exchangeable water” should be understood as meaning the water present in the crystals and which migrates to the surface of the latter during storage. The adipic acid crystals can also comprise what is known as included water, which originates from the inclusion of an amount of crystallization solution during the formation or treatment of the crystals. This water is not generally exchangeable, that is to say that it does not, without external action, migrate to the surface of the granule.

Another characteristic of the crystals of the invention is the total concentration of water in the granule, which must be greater than that of exchangeable water by at least 20 ppm. Preferably, this difference is between approximately 30 ppm and 2000 ppm, advantageously between 50 and 1000 ppm.

The amount of exchangeable water by weight of crystals is determined according to the following method:

An amount of powder or crystals to be analysed of approximately 300 g exactly weighed is placed in a hermetically closed container which is highly impermeable with respect to the external atmosphere. The volume of the container is 500 ml. A moisture-absorbing material composed of silica gel crystals (a weight of approximately 2 g weighed exactly) is also placed above or on the mass of powder or crystals to be analysed. This absorbing material is placed in a watch glass in order to avoid any contact between the various materials. The products to be analysed and the absorbing material are stored in the hermetically closed container for a period of time of 24 hours under the ambient conditions of temperature and pressure, that is to say at atmospheric pressure and at a temperature of between 5° C. and 25° C. approximately.

After 24 hours, the absorbing material is weighed. The difference in weight is corrected by the difference in weight obtained for a blank test, in which no powder or crystals to be analysed had been introduced into the container, carried out in parallel and under the same conditions as above.

The true difference in weight, that is to say after correction, is the same as the exchangeable water present in the powder or crystals to be analysed taken up by the absorbing material. It is expressed in ppm with respect to the mass of powder or crystals introduced into the container.

The total concentration of water in the crystals is determined according to the following analytical procedure:

The analyses are carried out in a Perkin-Elmer DSC7 cell with the following operating conditions:

rate of temperature rise: 5° C./min

sample carrier: crimped aluminium capsule

test sample: approximately 20 mg weighed exactly

fall in temperature to −50° C., followed by a rise to 25° C.

An endothermic peak following upon the rise in temperature appears at approximately 0° C., reproducing the solid/liquid transition of the water present in the crystals. The enthalpy difference measured translates into a total water content. The total water concentration is expressed in ppm with respect to the mass of crystals.

Another subject-matter of the present invention is a process for the manufacture of adipic acid crystals exhibiting the above characteristics.

This process consists in subjecting the adipic acid crystals obtained by crystallization to a maturing. This maturing consists in keeping the crystals at a temperature of between 10° C. and 80° C. in an atmosphere exhibiting an absolute humidity of less than 20 g/Sm³ for a period of time necessary to allow at least the majority of the exchangeable water present in the crystals to be removed. In other words, the maturing time is determined in order to obtain a concentration of exchangeable water in the crystals of less than 100 ppm, preferably of less than 50 ppm, as indicated above.

According to the invention, the process employs means for keeping the absolute humidity of the atmosphere in which the crystals are placed at a value of less than 20 g per Sm³ of gas, for example of air. The absolute humidity will advantageously be kept at a value of less than 10 g per Sm³ of air. Such means are, for example, means for absorbing moisture, such as hygroscopic products, for example silica gels, or the use of a dry gas, such as dry air, which is periodically or continually replaced.

Mention may be made, as examples of embodiments of the implementation of the maturing stage in accordance with the invention, of the storage of the crystals in a chamber with flushing of the head space of the chamber with a stream of dry air or sending, through the mass of the crystals, a stream of dry air. In the latter embodiment, the crystals can form a stationary bed through which the stream of dry air passes or a fluidized bed, if the speed of the air flow is sufficient to move the crystals.

It is also possible to carry out the stage of maturing the crystals by placing them in permeable packagings, that is to say packagings which allow at least the moisture present in the crystals to evaporate into the atmosphere surrounding the packagings or more advantageously which allow only the evaporation of moisture towards the outside of the packaging and which do not allow moisture to penetrate into the said packaging. The packagings can be positioned either in a closed chamber exhibiting an atmosphere with a controlled absolute humidity which is sufficiently low to allow evaporation of the exchangeable water present in the crystals or in an open chamber with replacement of the atmosphere in order for the latter to exhibit an absolute humidity which is sufficiently low to allow the evaporation of the exchangeable water.

Of course, it is possible to combine these various embodiments without, however, departing from the scope of the invention.

In addition, the description of various embodiments of the implementation of the maturing stage is given solely by way of illustration and has no limiting nature.

The adipic acid crystals treated by the process of the invention are generally obtained by crystallization from an aqueous adipic acid solution.

In addition, the processes for the manufacture of adipic acid generally comprise a stage of purification of the adipic acid which consists in crystallizing the acid from water. This crystallization can be carried out in a single crystallization stage or several successive crystallization stages.

The adipic acid recovered by filtration or centrifuging is in the form of irregularly shaped and reasonably large crystals. The distribution of the crystal sizes can be very broad as well as narrow.

The crystals can also be washed one or more times with water. Finally, the crystals are dried and then stored in receptacles or packagings for the purpose of transporting them to or feeding them on the site of their use.

According to the process of the invention, the adipic acid crystals are subjected after drying to the maturing stage described above. However, the dried crystals can be stored and subjected to the maturing stage before they are packaged in the transportation containers. The maturing stage can also be carried out during the transportation of the said crystals by use of suitable containers, for example a container which is impermeable with respect to external moisture but which makes possible the evaporation of the exchangeable water or which is kept in an atmosphere with a low absolute humidity. According to a preferred embodiment of the invention, the stage of maturing the crystals is carried out before the packaging of the latter for transportation, this packaging being carried out in impermeable containers to avoid uptake of moisture.

For this reason, the adipic acid crystals obtained by the process of the invention exhibit an excellent flowability and a very low possibility of caking. The containers can thus be emptied without difficulty, facilitating the control of the feeding of the adipic acid crystals in the various plants in which adipic acid is used.

It is therefore possible to store and transport these products for lengthy periods of time and under uncontrolled conditions with respect to the atmosphere.

The invention will be better illustrated in the light of the examples below, given solely by way [lacuna]:

### EXAMPLE 1

4 kg of adipic acid in the form of crystals with a mean size of 450 μm with a content of exchangeable water of 150 ppm, determined according to the procedure described above, are introduced into a 6 litre container. 5 g of dehydrating agent (silica gel) are placed in a watch glass above the bed. The container is impermeable. After maturing for 24 h at ambient temperature (20-25° C.), it is observed that the various batches treated do not cake, that is to say that, after storage in a closed container for several weeks, no agglomeration of crystals with one another was observed. The content of exchangeable water in the treated crystals, determined by the test described above, is less than 10 ppm and the total concentration of water is 900 ppm.

### COMPARATIVE EXAMPLE 2

A storage test in an impermeable container on the adipic acid described above which has not been subjected to any maturing shows numerous agglomerations of crystals with one another, preventing the crystals from being easily withdrawn from the storage container.

### EXAMPLE 3

An amount of adipic acid of 1.4 kg in the form of crystals with a mean size of 330 μm with a content of exchangeable water of 150 ppm, determined according to the procedure described above, is introduced into a closed glass column (diameter 75 mm, height 1 m). After storing for a few hours, the crystals agglomerated and it is impossible to empty the column by natural flow of the crystals.

The product present in the column is kept in the column for approximately 15 hours and is then subjected to fluidization with dry air for a few minutes. After keeping this treated acid in an impermeable storage container for several weeks, no agglomeration of crystals is observed. The content of exchangeable water according to the procedure described above is less than 10 ppm and the total concentration of water is 920 ppm.

### EXAMPLE 4

500 kg of adipic acid crystals with a mean size of 420 μm and with a content of exchangeable water of 130 ppm, determined according to the above procedure, are bagged up in a permeable bag (woven bag) and are left under ambient conditions of temperature (20-25° C.) and of pressure (the external relative humidity varies according to the temperature from 40 to 50%) [indicate the values in absolute humidity].

Humidity sensors placed in the product allow the relative humidity in the atmosphere of the product to be monitored. After storage for a few hours, an equilibrium between the external absolute humidity and the absolute humidity inside the bag is observed. Under these storage conditions, no agglomeration of crystals is observed. The content of exchangeable water in these crystals is less than 20 ppm and the total concentration of water is 910 ppm.

In contrast, the storage in impermeable bags under the same conditions of temperature and humidity results in significant agglomeration. The analysis of the content of exchangeable water in these crystals shows that the content has not varied.

### EXAMPLE 5

Carrying out the maturing of and the removal of exchangeable water (by flushing with dry gas) from a batch of adipic acid with a mean size of 320 μm which initially contains 120 ppm of exchangeable water according to the procedure described in this patent.

20 T of adipic acid crystals with a mean size of 320 μm exhibiting a concentration of exchangeable water of 120 ppm are bagged up in a shipping container having a bag which is not very permeable to the moisture in gases (less than 1 g/m²/d). The top of the shipping container (with a surface area of 20 m²) is flushed with a dry gas (400 Sm³/h) for a period of 10 days. After halting this treatment and closing the bag, the product does not cake and the shipping container can be easily emptied (in less than one hour) after transportation for 2 months. The absolute humidity during the transportation was less than 10 g/m³, the content of exchangeable water in the crystals on exiting from the shipping container was less than 10 ppm and the total concentration of water was 890 ppm.

1-14. (Canceled).

15. Adipic acid crystals, having a concentration of exchangeable water less than 100 ppm, said concentration being determined from 300 g of adipic acid crystals placed in a hermetic chamber, purged beforehand with dry air, with 2 g of a moisture-absorbing material, said chamber being kept at a temperature of between 5° C. and 25° C. for 24 hours, the concentration of water being equal to the amount of water absorbed by the absorbing material relative to 1 g of crystals, and the total concentration of water being greater than that of exchangeable water by at least 20 ppm.

16. The crystals according to claim 15, wherein the abovementioned concentration of water is less than 50 ppm.

17. The crystals according to claim 15, having a difference between the total concentration of water and that of exchangeable water of between 30 ppm and 2000 ppm.

18. A process for the manufacture of adipic acid crystals as defined in claim 15, comprising the step of subjecting the adipic acid crystals obtained by crystallization in an aqueous medium or treated with an aqueous solution to a maturing stage consisting in keeping the crystals at a temperature of between 10° C. and 80° C. for a period of time necessary to obtain a concentration of exchangeable water of less than 100 ppm in the presence of means for keeping the absolute humidity in the atmosphere around the crystals at a value of less than 20 g/Sm³.

19. The process according to claim 18, comprising the step of placing the crystals in a chamber with a means for lowering to or maintaining at a value of less than 20 g/Sm³ the absolute humidity of the atmosphere of the chamber for a period of time sufficient to obtain a constant absolute humidity in the said atmosphere.

20. The process according to claim 19, wherein the means for maintaining or lowering the absolute humidity of the atmosphere is a means for continually or periodically replacing the atmosphere surrounding the crystals with an atmosphere with an absolute humidity of less than 20 g/Sm³.

21. The process according to claim 20, wherein said replacement is obtained by flushing with a stream of gas with an absolute humidity of less than 20 g/Sm³.

22. The process according to claim 21, wherein the stream of gas is fed through the mass of crystals.

23. The process according to one of claim 21, wherein the gas is dry air.

24. The process according to claim 19, wherein the means for maintaining or lowering the absolute humidity of the atmosphere comprises a device for absorbing the said humidity positioned in the chamber.

25. The process according to claim 24, wherein the absorption device comprises a moisture-absorbing compound.

26. The process according to one of claims 19, wherein the means for lowering or maintaining the humidity further comprises a means for replacing the atmosphere and a moisture-absorbing device.

27. The process according to claim 19, wherein the chamber comprising the crystals comprises a covering permeable from the inside outwards and impermeable from the outside inwards.

28. The process according to one of claim 19, wherein the maturing stage is carried out before the packaging of the adipic acid crystals in shipping containers which are impermeable to the moisture in the surrounding air for storage or transportation.
